(19) United States
(12) Patent Application Publication
(10) Pub. No.: US 2006/0121486 A1
(43) Pub. Date: Jun. 8, 2006

(54) Haplotype partitioning

(75) Inventors: David N. Cooper, Cardiff (GB); Michael Krawczak, Kiel (DE); Jurgen Hedderich, Kiel (DE)

Correspondence Address: KING & SCHICKLI, PLLC, 247 NORTH BROADWAY, LEXINGTON, KY, 40507, US

(73) Assignee: DAVID N. COOPER ETAL

(21) Appl. No.: 10/539,953

(22) PCT Filed: Dec. 11, 2003
(86) PCT No.: PCT/GB03/05412

§ 371 Date: Jan. 9, 2006

(30) Foreign Application Priority Data
Dec. 19, 2002 (GB) 0229725.7

(51) Int. Cl. C12Q 1/68; G06F 19/00

(52) U.S. Cl. 435/6; 702/20

(57) ABSTRACT

The invention relates to a method for identifying mutations and/or polymorphisms that are major determinants of a selected phenotype and is based on the identification of haplotypes and the partitioning thereof into groups that are major determinants for said phenotype.

The invention relates to a novel method for determining the significance of polymorphisms or mutations in at least one gene; and the significant polymorphisms or mutations identified thereby.

Since the advent of gene sequencing technology in the late-1980's, and the establishment of The Human Genome Project, an enormous amount of information has been discovered about the sequence structure, or nature, of a vast variety of genes, especially in man. Moreover, as gene sequencing methods have evolved there has been an increase in the number of variations detected within any given gene. Given that a typical gene could be 30 kilobases in length and that variations occur on average every 1100 bases, it follows that a tremendous amount of work needs to be undertaken in order to determine which variants are of clinical or technological significance. However, this is a prerequisite step if one is to exploit the knowledge available.

Some genes are more subject to variations than others. Highly polymorphic genes provide a particular challenge to researchers who need to determine which variation at a given site in a nucleic acid molecule, or which combination of variations at given sites within the nucleic acid molecule, is/are significant. It follows that within any given population, the study of a single gene from a number of organisms, or individuals, may produce a considerable amount of information because where a plurality of polymorphic sites are present in a given gene the polymorphic characteristics may vary from individual to individual. Accordingly, when a number of polymorphic sites are investigated a pattern, or signature, that is characteristic of each individual is produced. This is known as the haplotype. Each haplotype represents a particular combination of variations at a plurality of polymorphic sites. It is therefore the job of the skilled researcher to sift through haplotypes in order to determine which are significant. As the skilled reader will appreciate this is a long, difficult and, often, tedious task. It can involve studying various properties of the gene, or the protein encoded thereby, in order to determine what, if any, are the implications of each haplotype.

With this in mind, we have developed a methodology which facilitates the study of genetic variations. Our methodology is directed towards examining a number of variations within a gene and determining the significance thereof. More specifically, our methodology is directed towards looking at a plurality of variations at a plurality of polymorphic sites in at least one gene in order to determine the significance thereof. Essentially, our methodology can be used to examine the relative significance of difference haplotypes. It therefore, effectively, sifts through a plurality of haplotypes in order to determine which are the most significant. It therefore has the ability to partition a vast amount of data in order to select the most relevant forms thereof.

Human stature is a highly complex trait resulting from the interaction of multiple genetic and environmental factors. Since familial short stature is already known to be associated with inherited mutations of the growth hormone gene, it is reasonable to assume that polymorphic variations in this pituitary-expressed gene influence adult height. It is known that there are a considerable number of polymorphic variations within this gene and, indeed, the proximal region of the GH1 growth hormone gene promoter exhibits a high level of sequence variation with 16 single nucleotide polymorphisms reported within a 535 base-pair stretch. The majority of these SNPs occur at the same positions in which the GH1 gene differs from the paralogous GH2, CSH1, CSH2 and CSHP1 genes located in the cluster of five genes that contain GH1. These five genes are located on chromosome 17q23 as a 66 kb cluster.

Moreover, the expression of human GH1 gene is also influenced by a Locus Control Region (LCR) located between 14.5 kb and 32 kb upstream of the GH1 gene. The LCR contains multiple DNase I hypersensitive sites and is required for the activation of the genes of the GH1 gene cluster in both pituitary and placenta.

Accordingly, given the high level of variation within this gene we have used it to develop our methodology. More specifically we have used this gene to assess the relative importance of polymorphic variation in both the proximal promoter region and the LCR region of GH1 gene expression.

### STATEMENT OF THE INVENTION

We describe herein a method of haplotype partitioning to identify mutations and/or polymorphisms that are major determinants of phenotype, particularly, but not exclusively, phenotype that is either advantageous or disadvantageous. For example, perhaps most typically, the method will be used to identify mutations and/or polymorphisms that are responsible, wholly or in part, for a physiological condition or disorder, such as, for example, a disease or abnormal or undesirable state.

Accordingly, the method of haplotype partitioning of the invention comprises examining the residual deviance (δ) for each selected group of mutations and/or polymorphisms of a gene under consideration.

More ideally the method comprises examining the residual deviance (δ) of possible subsets of mutations and/or polymorphisms and so, most advantageously, the method is undertaken to examine the residual deviance (δ), of the partitioning of haplotypes {1 . . . m}, based on each possible subset of mutations and/or polymorphisms.

Most ideally still the method involves using the following function
δ=δ(Π)=Σᵢ₌₁^{m}(χᵢ−\overline{χ}_{Π(i)})²
(See pages 18 and 22 for definitions)

The method of the invention is applicable, but not exclusively, to situations where the effects of said mutations and/or polymorphisms are strongly interdependent such as, for example, in the instance where there is linkage disequilibrium.

Using this methodology it is possible to identify those mutations and/or polymorphisms that are responsible for a sizeable proportion of the residual deviance in, for example, expression levels (where the mutations and/or polymorphisms are in the promoter region of the gene) or, for example, protein function (where the mutations and/or polymorphisms are in the protein coding sequence of the gene).

Advantageously the methodology of the invention can be used to predict, and so subsequently make, super-maximal and sub-minimal haplotypes which may be useful, for example, as experiment controls in subsequent testing programmes.

Other methods for the identification of mutations and/or polymorphisms responsible for a sizeable proportion of the phenotype under consideration are described herein and constitute various aspects and/or embodiments of the invention.

According to a further aspect of the invention there is described herein significant mutations and/or polymorphisms, in the form of single nucleotide polymorphisms (SNPs), that are major determinants of at least one selected phenotype.

More specifically, these SNPs can be located in the proximal promoter of at least one selected gene and so determine the level of expression of corresponding protein and so the likely selected phenotype of an individual.

It follows that knowledge of these SNPs or this subset of SNPs has utility in diagnostic techniques.

According to a further aspect of the invention there is provided a detection method for detecting a haplotype effective to act as an indicator of at least one phenotype in an individual, which detection method comprises the steps of:
(a) obtaining a test sample of genetic material from an individual to be tested, said material comprising, at least, a selected gene or a fragment thereof; and
(b) analysing the nucleotide sequence of said gene, or fragment thereof, to see if any single nucleotide polymorphisms exist at any one or more of the SNP sites within the gene;
(c) where said SNPs exist, identifying them and subjecting them to analysis using the aforementioned method.

A man skilled in the art will appreciate that the afore methodology can be undertaken at, or in, one or more regions of a gene either, N terminal in order to determine the effects of polymorphic variation within a promoter or within the coding region in order to determine the effects of polymorphic variation on the protein.

Moreover, the methodology of the invention has use in determining a super-maximal and sub-minimal haplotype and therefore the invention, according to a further aspect, also comprises the identification of a super-maximal and/or sub-minimal haplotype for at least one gene.

In the examples given herein the super-maximal haplotype for the growth hormone gene is defined by the following coding sequence: AGGGGTTAT-ATGGAG at SNP−476, −364, −339, −308, −301, −278, −168, −75, −57, −31, −6, −1, +3, +16, +25, +59, relative to GH1 gene transcriptional start site. Conversely, the sub-minimal haplotype is defined as the following coding sequence with respect to the same site: AG-TTTTGGGGCCACT.

According to a further aspect of the invention there is provided at least one haplotype identified by the aforementioned methodology and more specifically there is provided the use of said haplotype in the diagnosis or treatment of a given disease or in the development of a super-expression protein.

Reference herein to the term super-expression includes reference to the over expression of a given protein with respect to the wild-type.

The methodology of the invention will now be described by way of the following information which concerns the materials and methods that were undertaken to identify various haplotypes, provide for their partitioning, and assess their functional significance.

### FIGURE LEGENDS

FIG. 1: GH1 gene promoter expression of negative controls as measured on different plates (a), and normalized expression levels of the wild-type haplotype (1), displayed as multiples of the plate-wise mean expression level of the wild-type (b).

FIG. 2: Location of 16 SNPs in the GH1 promoter relative to the transcriptional start site (denoted by an arrow). The hatched box represents exon 1. The positions of the binding sites for transcription factors, nuclear factor 1 (NF1), Pit-1 and vitamin D receptor (VDRE), the TATA box and the translational initiation codon (ATG) are also shown.

FIG. 3: Normalized expression levels of the 40 GH1 haplotypes relative to the wild-type (haplotype 1). Haplotypes associated with a significantly reduced level of luciferase reporter gene expression (by comparison with haplotype 1) are denoted by hatched bars. Haplotypes associated with a significantly increased level of luciferase reporter gene expression (by comparison with haplotype 1) are denoted by solid bars. Haplotypes are arranged in decreasing order of prevalence.

FIG. 4: Minimum relative residual deviance δ_{R}(Π_{k,min}) of normalized expression levels associated with haplotype partitioning using k SNPs (shaded bars). The dotted curve depicts the number of haplotypes comprising the minimum-δ_{R}-partitioning Π_{k,min}.

FIG. 5: Relationship between size and cross-validated δ_{R} value for minimum deviance intermediate trees, using six selected SNPs (nos. 1, 6, 7, 9, 11 and 14). The dotted (horizontal) line corresponds to one SE of the cross-validated δ_{R} of the fully grown tree; the dashed (vertical) line indicates the smallest tree for which the cross-validated δ_{R} lies within one SE of that of the fully grown tree.

FIG. 6: Regression tree of GH1 gene promoter expression as obtained by recursive binary haplotype partitioning, using six selected SNPs (nos. 1, 6, 7, 9, 11 and 14). Numbers on nodes refer to the SNPs by which the respective nodes were split. Terminal nodes (‘leaves’) are depicted as squares and numbered from left to right.

FIG. 7: ‘Reduced Median Network’ connecting the seven haplotypes (circles) that have been observed at least 8 times in 154 male Caucasians. The size of each circle is proportional to the frequency of the respective haplotype in the control sample. Haplotypes H12 and H23 have been included as connecting nodes even although they have been observed only 5 and 2 times, respectively. SNPs at which haplotypes differ are given alongside each branch. The dark dot marks a non-observed haplotype or a double mutation at SNP sites 4 and 5.

FIG. 8: Differences in protein binding capacity between GH1 promoter SNP alleles revealed by electrophoretic mobility shift (EMSA) assays. Arrows denote allele-specific interacting proteins. The arrowhead denotes the position of a Pit-1-like binding protein. −ve (negative control), +ve (positive control), S (specific competitor), N (non-specific competitor), P (Pit-1 consensus sequence), P* (prolactin gene Pit-1 binding site), TSS (transcriptional initiation site).

### MATERIALS AND METHODS

Human Subjects

DNA samples were obtained from lymphocytes taken from 154 male British army recruits of Caucasian origin who were unselected for height. Height data were available for 124 of these individuals (mean, 1.76±0.07 m) and the height distribution was found to be normal (Shapiro-Wilk statistic W=0.984, p=0.16). Ethical approval for these studies was obtained from the local Multi-Regional Ethics Committee.

Polymerase Chain Reaction (PCR) Amplification

PCR amplification of a 3.2 kb GH1 gene-specific fragment was performed using oligonucleotide primers GH1F (5′ GGGAGCCCCAGCAATGC 3′; −615 to −599) and GH1R (5′ TGTAGGAAGTCTGGGGTGC 3′; 2598 to 2616) [numbering relative to the transcriptional initiation site at +1 (GenBank Accession No. J03071)]. A 1.9 kb fragment containing sites I and II of the GH1 LCR was PCR amplified with LCR5A (5′ CCAAGTACCTCAGATGCAAGG 3′; −315 to −334) and LCR3.0 (5′ CCTTAGATCTTGGCCTAGGCC 3′; 1589 to 1698) [LCR sequence was obtained from GenBank (Accession No. AC005803) whilst LCR numbering follows that of Jin et al. 1999; GenBank (Accession No. AF010280)]. Conditions for both reactions were identical; briefly, 200 ng lymphocyte DNA was amplified using the Expand™ high fidelity system (Roche) using a hot start of 98° C. 2 min, followed by 95° C. 3 min, 30 cycles 95° C. 30 s, 64° C. 30 s, 68° C. 1 min. For the last 20 cycles, the elongation step at 68° C. was increased by 5 s per cycle. This was followed by further incubation at 68° C. for 7 min.

Cloning and Sequencing

Initially, PCR products were sequenced directly without cloning. The proximal promoter region of the GH1 gene was sequenced from the 3.2 kb GH1-specific PCR fragment using primer GH1S1 (5′ GTGGTCAGTGTTGGAACTGC 3′: −556 to −537). The 1.9 kb GH1 LCR fragment was sequenced using primers LCR5.0 (5′ CCTGTCACCTGAGGATGGG 3′; 993 to 1011), LCR3.1 (5′ TGTGTTGCCTGGACCCTG 3′; 1093 to 1110), LCR3.2 (5′ CAGGAGGCCTCACAAGCC 3′; 628 to 645) and LCR3.3 (5′ ATGCATCAGGGCAATCGC 3′; 211 to 228). Sequencing was performed using BigDye v2.0 (Applied Biosystems) and an ABI Prism 377 or 3100 DNA sequencer. In the case of heterozygotes for promoter region or LCR variants, the appropriate fragment was cloned into pGEM-T (Promega) prior to sequencing.

Construction of Luciferase Reporter Gene Expression Vectors

Individual examples of 40 different GH1 proximal promoter haplotypes (Table 1) were PCR amplified as 582 bp fragments with primers GHPROM5 (5′ AGATCTGACCCAGGAGTCCTCAGC 3′; −520 to −501) and either GHPROM3A (5′ AAGCTTGCAGCTAGGTGAGCTGTC 3′; 44 to 62) or GHPROM3C (5′ AAGCTTGCCGCTAGGTGAGCTGTC 3′; 44 to 62) depending on the base at position +59 of the haplotype. To facilitate cloning, all primers had partial or complete non-templated restriction endonuclease recognition sequences added to their 5′ ends (denoted in bold above); Bg/II (GHPROM5) and HindIII (GHPROM3A and GHPROM3C). PCR fragments were then cloned into pGEM-T. Plasmid DNA was initially digested with HindIII (New England Biolabs) and the 5′ overhang removed with mung bean nuclease (New England Biolabs). The promoter fragment was released by digestion with Bg/II (New England Biolabs) and gel purified. The luciferase reporter vector pGL3 Basic was prepared by Ncol (New England Biolabs) digestion and the 5′ overhang removed with mung bean nuclease. The vector was then digested with Bg/II (New England Biolabs) and gel purified. The restricted promoter fragments were cloned into luciferase reporter gene vector GL3 Basic. Plasmid DNAs (pGL3GH series) were isolated (Qiagen midiprep system) and sequenced using primers RV3 (5′ CTAGCAAAATAGGCTGTCCC 3′; 4760 to 4779), GH1SEQ1 (5′ CCACTCAGGGTCCTGTG 3′; 27 to 43), LUCSEQ1 (5′ CTGGATCTACTGGTCTGC 3′; 683 to 700) and LUCSEQ2 (5′ GACGAACACTTCTTCATCG 3′; 1372 to 1390) to ensure that both the GH1 promoter and luciferase gene sequences were correct. A truncated GH1 proximal promoter construct (−288 to +62) was also made by restriction of pGL3GH1 (haplotype 1) with Ncol and Bg/II followed by blunt-ending/religation to remove SNP sites 1-5.

Artificial proximal promoter haplotype reporter gene constructs were made by site-directed mutagenesis (SDM) [Site-Directed Mutagenesis Kit (Stratagene)] to generate the predicted super-maximal haplotype (AGGGGTTAT-ATGGAG) and sub-minimal haplotypes (AG-TTGTGGGACCACT and AG-TTTTGGGGCCACT).

To make the LCR-proximal promoter fusion constructs, the 1.9 kb LCR fragment was restricted with Bg/II and the resulting 1.6 kb fragment cloned into the Bg/II site directly upstream of the 582 bp promoter fragment in pGL3. The three different LCR haplotypes were cloned in pGL3 Basic, 5′ to one of three GH1 proximal promoter constructs containing respectively a “high expressing promoter haplotype” (H27), a “low expressing promoter haplotype” (H23) and a “normal expressing promoter haplotype” (H1) to yield a total of nine different LCR-GH1 proximal promoter constructs (pGL3GHLCR). Plasmid DNAs were then isolated (Qiagen midiprep) and sequence checked using appropriate primers.

Luciferase Reporter Gene Assays

In the absence of a human pituitary cell line expressing growth hormone, rat GC pituitary cells (Bancroft 1973; Bodner and Karin 1989) were selected for in vitro expression experiments. Rat GC cells were grown in DMEM containing 15% horse serum and 2.5% fetal calf serum. Human HeLa cells were grown in DMEM containing 5% fetal calf serum. Both cell lines were grown at 37° C. in 5% CO₂. Liposome-mediated transfection of GC cells and HeLa cells was performed using Tfx™-20 (Promega) in a 96-well plate format. Confluent cells were removed from culture flasks, diluted with fresh medium and plated out into 96-well plates so as to be ˜80% confluent by the following day.

The transfection mixture contained serum-free medium, 250 ng pGL3GH or pGL3GHLCR construct, 2 ng pRL-CMV, and 0.5 μl Tfx™-20 Reagent (Promega) in a total volume of 90 μl per well. After 1 hr, 200 μl complete medium was added to each well. Following transfection, the cells were incubated for 24 hrs at 37° C. in 5% CO₂ before being lysed for the reporter assay.

Luciferase assays were performed using the Dual Luciferase Reporter Assay System (Promega). Assays were performed on a microplate luminometer (Applied Biosystems) and then normalized with respect to Renilla activity. Each construct was analysed on three independent plates with six replicates per plate (i.e. a total of 18 independent measurements). For the proximal promoter assays, each plate included negative (promoterless pGL3 Basic) and positive (SV40 promoter-containing pGL3) controls. For the LCR analysis, constructs containing the proximal promoter but lacking the LCR were used as negative controls.

Electrophoretic Mobility Shift Assay (EMSA)

EMSA was performed on double stranded oligonucleotides that together covered all 16 SNP sites (Table 2). Nuclear extracts from GC and HeLa cells were prepared as described by Berg et al. (1994). Oligonucleotides were radiolabelled with [γ−³³P]-dATP and detected by autoradiography after gel electrophoresis. EMSA reactions contained a final concentration of 20 mM Hepes pH7.9, 4% glycerol, 1 mM MgCl₂, 0.5 mM DTT, 50 mM KCl, 1.2 μg HeLa cell or GC cell nuclear extract, 0.4 μg poly[dl-dC].poly[dl-dC], 0.4 pM radiolabelled oligonucleotide, 40 pM unlabelled competitor oligonucleotide (100-fold excess) where appropriate, in a final volume of 10 μl. EMSA reactions were incubated on ice for 60 mins and electrophoresed on 4% PAGE gels at 100V for 45 mins prior to autoradiography. For each reaction, a double stranded unlabelled test oligonucleotide was used as a specific competitor whilst an oligonucleotide derived from the NF1 gene promoter (5′ CCCCGGCCGTGGAAAGGATCCCAC 3′) was used as a non-specific competitor. Double stranded oligonucleotides corresponding to the human prolactin (PRL) gene Pit-1 binding site (5′ TCATTATATTCATGAAGAT 3′) and the Pit-1 consensus binding site (5′ TGTCTTCCTGAATATGAATAAGAAATA 3′) were used as specific competitors for protein binding to the SNP 8 site.

Primer Extension Assays

Primer extension assays were performed to confirm that constructs bearing different SNP haplotypes utilized identical transcriptional initiation sites. Primer extension followed the method of Triezenberg et al. (1992).

Data Normalization

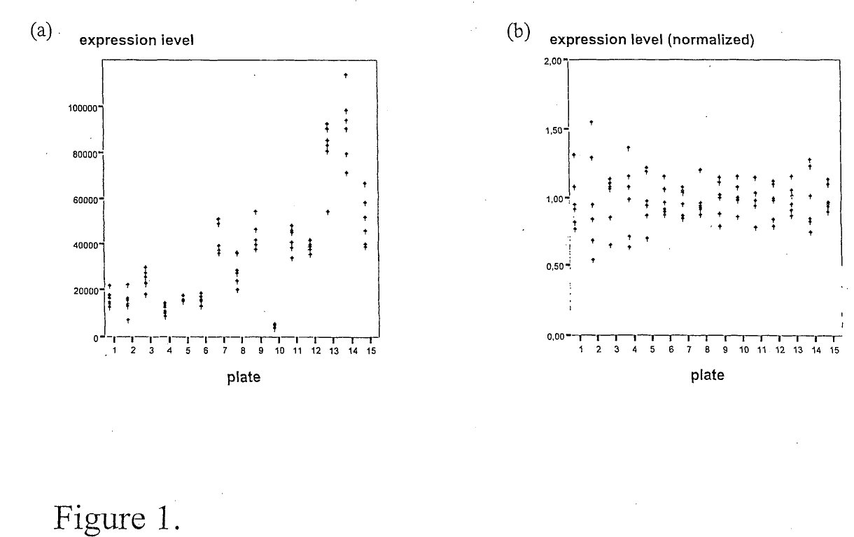

Expression measurements for negative controls (promoterless pGL3 Basic) exhibited considerable variation between plates (FIG. 1a). To correct the data for base-line expression and plate effects, the mean activity of the negative controls on a given plate was subtracted from all other activity values on the same plate. The mean (plate-corrected) activity for proximal promoter haplotype 1 (H1) on each plate was then calculated, and all other haplotype-associated activities on the same plate were divided by this value. These two transformations ensured that the mean negative control activity equalled zero whilst the mean activity of H1 equalled unity, independent of plate number. Resulting activity values may thus be interpreted as fold changes in comparison to H1, corrected for both baseline and plate effects. Since no significant plate effect was detectable after transformation, the data were combined over plates. The results of this normalization procedure are illustrated for H1 in FIG. 1b. A procedure similar to that used for the analysis of the proximal promoter haplotypes was also followed for the LCR-promoter fusion construct expression data, using haplotype A as the reference haplotype.

Statistical Analysis

Normalized expression levels of the proximal promoter haplotypes were tested for goodness-of-fit to a Gaussian distribution using the Shapiro-Wilk statistic (W) as implemented in procedure UNIVARIATE of the SAS statistical analysis software (SAS Institute Inc., Cary N.C., USA). Significance assessment was adjusted for multiple (i.e. 40-fold) testing by setting p_{critical}=0.05/40≈0.001. Using this criterion, the expression levels of two promoter haplotypes were found to differ significantly from a Gaussian distribution viz. H21 (W=0.727, p=0.0002) and H40 (W=0.758, p=0.0004). For the other 38 haplotypes, expression levels were regarded as consistent with normality and were therefore subjected to pair-wise comparison using Tukey's studentized range test (SAS procedure GLM). Pair-wise comparison of expression levels between groups of different haplotypes was performed using normal approximation z of the Wilcoxon rank sum statistic (SAS procedure NPAR1WAY).

The SNPs analysed in this study exerted their influence upon proximal promoter expression in a complex and highly interactive fashion. Further, owing to linkage disequilibrium, expression levels associated with individual polymorphisms were found to be strongly interdependent. It was thus expected that a substantial proportion of the observed variation in expression level would be attributable to variation at a small subset of polymorphic sites. In order to assess formally the correlation structure between the SNPs, and to be able to identify an appropriate subset of critical polymorphisms for further study, the residual deviance upon haplotype partitioning was calculated for all possible subsets of proximal promoter SNPs.

For a given partitioning {1 . . . m}=Π=π₁∪ . . . ∪πₖ of a set of data points x₁, . . and with π(i)=j if i∈πⱼ, the residual deviance δ of Π is defined as
δ=δ(Π)=Σᵢ₌₁^{m}(χᵢ−\overline{χ}_{Π(i)})².

When the data set was not partitioned at all, then δ=δ(Π₀)=421.7, and the relative residual deviance of any other partitioning Π was defined as δ_{R}(Π)=δ(Π)/δ(Π₀).

Six SNPs (nos. 1, 6, 7, 9, 11 and 14; see below) were identified as being responsible for a sizeable proportion (˜60%) of the residual deviance in expression level at the same time as invoking relatively little haplotype variation. The statistical interdependence of these SNPs was further analysed by means of a regression tree, constructed by recursive binary partitioning using statistics software R (Ihaka and Gentleman 1996). In the tree construction process, the SNPs were used individually as predictor variables at each node so as to select the two most homogeneous subgroups of haplotypes with respect to the response variable (i.e. normalized proximal promoter expression). The node and SNP that served to introduce a new split were chosen so as to minimize äR for the partitioning as defined by the terminating nodes (‘leaves’) of the resulting intermediate tree. This process was continued until all leaves corresponded to individual haplotypes (‘fully grown tree’). The reliability of the δ_{R} estimates was assessed in each step by 10-fold cross-validation and the standard error (SE) was calculated.

Regression analysis of height and proximal promoter expression level in vitro was performed for the 124 height-known individuals studied using the CANCORR procedure of the SAS software package. Let μ_{nor,h1} and μ_{nor,h2} denote the mean normalized expression levels of the two haplotypes carried by a given individual. The height of individuals not homozygous for H1 (n=109) was modelled as

$$height = {α}_{0} + {α}_{1} \cdot \frac{{μ}_{nor , h1} + {μ}_{nor , h2}}{2} + {α}_{2} \frac{\cdot {μ}_{nor , h1}^{2} + {μ}_{nor , h2}^{2}}{2} + {α}_{3} \cdot {μ}_{nor , h1} \cdot {μ}_{nor , h2}$$

and the coefficient of determination, r², calculated.

A reduced median network (Bandelt et al. 1995) was constructed for the seven promoter haplotypes (H1-H7) that were observed at least 8 times in the 154 study individuals.

Linkage Disequilibrium Analysis

Linkage disequilibrium (LD) between promoter SNPs, and between SNPs and LCR haplotypes, was evaluated in 100 individuals randomly chosen from the total of 154 under study, using parameter ρ as devised for biallelic loci by Morton et al. (2001). Whilst ρ=1 is equivalent to two loci showing complete LD, ρ=0 indicates complete lack of LD. Only eight SNPs were found to be sufficiently polymorphic in the population sample (heterozygosity ᵢ Y5%) to warrant inclusion. SNP5 was excluded owing to its perfect LD with SNP4 (only two pair-wise haplotypes present). Maximum likelihood estimates of the combined LCR-proximal promoter haplotype frequencies, as required for LD analysis, were obtained using an in-house implementation of the expectation maximization (EM) algorithm.

Results

Proximal Promoter Polymorphism Frequencies and Haplotypes

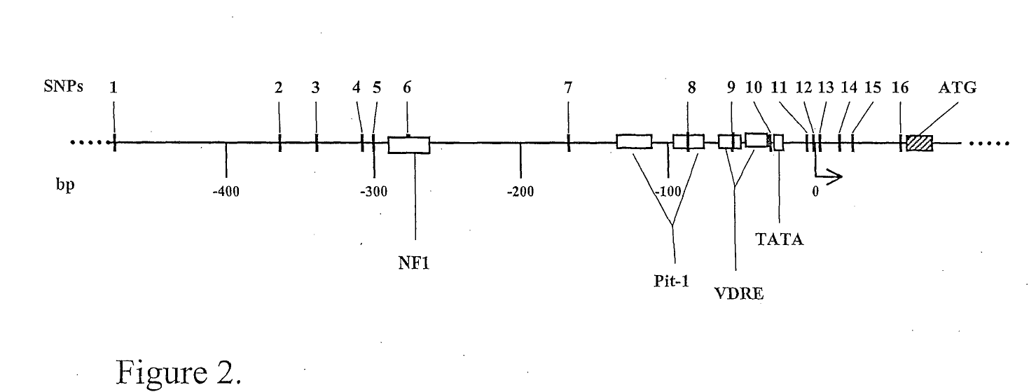

The GH1 gene promoter region has been reported to contain 16 polymorphic nucleotides within a 535 bp stretch (Table 3; Giordano et al. 1997; Wagner et al. 1997). These SNPs were enumerated 1-16 for ease of identification (FIG. 2). In a study of 154 male British Caucasians, 15 of these SNPs (all except no. 2) were found to be polymorphic (minor allele frequencies 0.003 to 0.41; Table 3). Variation at the 16 positions was ascribed to a total of 36 different promoter haplotypes (Table 1). Haplotype 1 (H1) may thus be described by a sequence of 16 bases (GGGGGGTATGAAGAAT), representing the 16 SNP locations from −476 to +59. The frequency of the 36 promoter haplotypes varied from 0.339 for H1, henceforth referred to as ‘wild-type’, to 0.0033 (nos. 25-36) (Table 1). A further 4 haplotypes (nos. 37-40) were found as part of a separate study in 4 individuals exhibiting short stature (Table 1). These haplotypes were absent from the study group but were included in the subsequent analysis for the sake of completeness.

Proximal Promoter Haplotypes and Relative Promoter Strength

The 40 promoter haplotypes were studied by in vitro reporter gene assay and found to differ with respect to their ability to drive luciferase gene expression in rat pituitary cells (Table 4). Expression levels were found to vary over a 12-fold range with the lowest expressing haplotype (no. 17) exhibiting an average level that was 30% that of wild-type and the highest expressing haplotype (no. 27) exhibiting an average level that was 389% that of wild-type (Table 4). Twelve haplotypes (nos. 3, 4, 5, 7, 11, 13, 17, 19, 23, 24, 26 and 29) were associated with a significantly reduced level of luciferase reporter gene expression by comparison with H1. Conversely, a total of 10 haplotypes (nos. 14, 20, 27, 30, 34, 36, 37, 38, 39 and 40) were associated with a significantly increased level of luciferase reporter gene expression by comparison with H1 (Table 4). Constructs bearing different SNP haplotypes were shown by primer extension assay to utilize identical transcriptional initiation sites (data not shown). Expression of the reporter gene constructs was found to be 1000-fold lower in HeLa cells than in GC cells (data not shown).

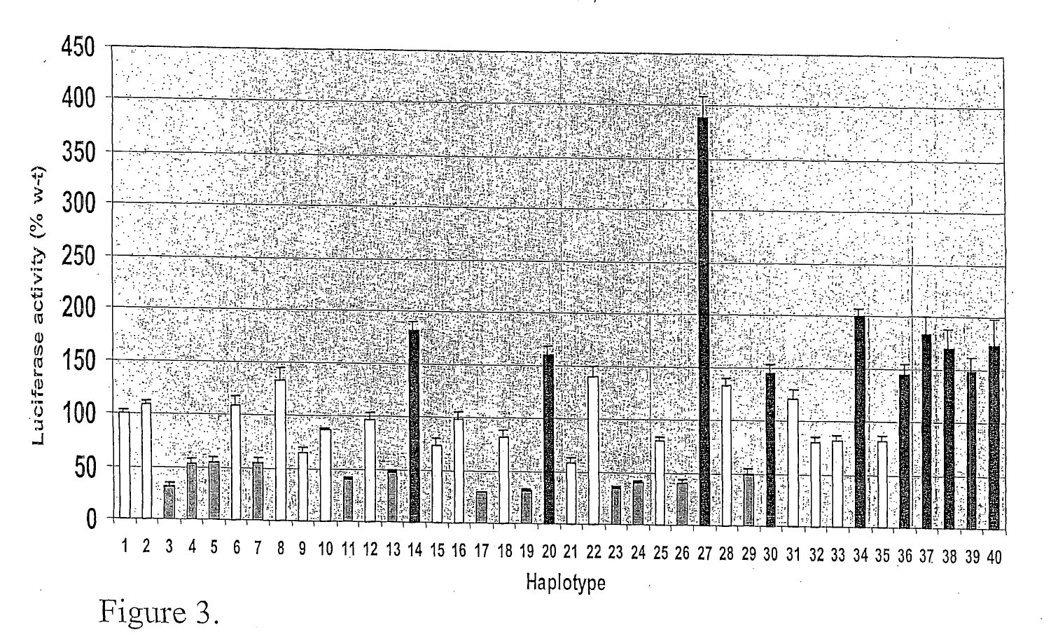

The in vitro expression levels of the 40 different GH1 promoter haplotypes are presented graphically in FIG. 3. A tendency is apparent for the low expressing haplotypes to occur more frequently whereas the high expressing haplotypes tend to occur less frequently (Wilcoxon P<0.01). Since this finding is suggestive of the action of selection, selection effects were sought at the level of individual SNPs. For the 15 SNPs studied here, the mean expression level (weighted by haplotype frequency) and the frequency of the rarer allele in controls were found to be positively correlated (Spearman rank correlation coefficient, r=0.32). If SNP 7 is excluded as an outlier (it has a particularly high expression level associated with the rarer allele), r=0.53 with a one sided p<0.05.

The in vitro expression level associated with the truncated promoter construct lacking SNPs 1-5 was 102±5% that of the wild-type (haplotype 1). Thus it may be inferred that SNPs 1-5 are likely to have a limited direct influence on GH1 gene expression.

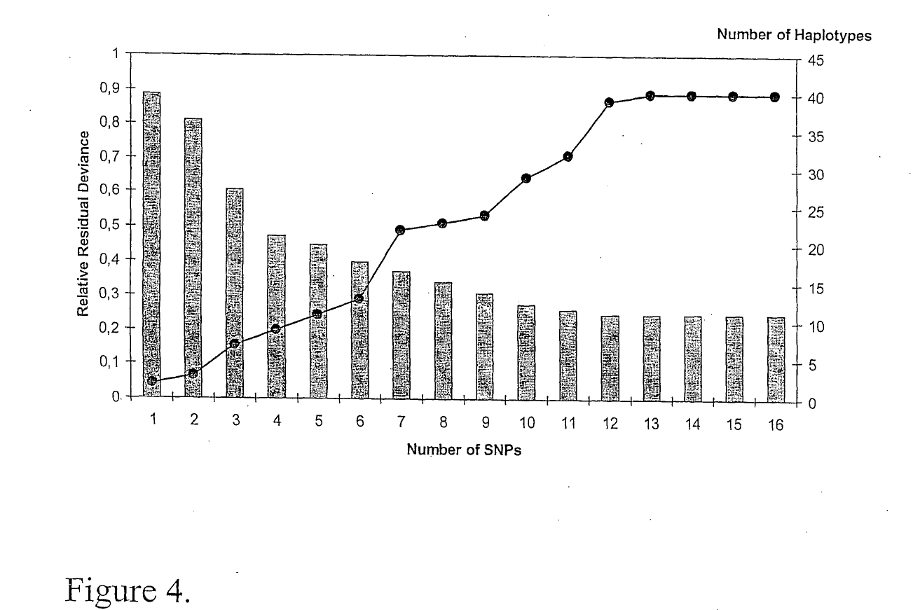

Expression levels associated with individual SNPs were found to be strongly interdependent. An attempt was therefore made to partition the expression data in such a way as to identify a subset of key polymorphic sites that contribute disproportionately to the observed variation in in vitro expression level. Partitioning by the full haplotype comprising all 16 SNPs yielded a relative residual deviance of δ_{R}(Π₁₆)=0.245. This can be interpreted in terms of 24.5% of the variation in expression level not being accountable by variation in haplotype. For 1≦k<16, the minimum-δ_{R}-partitioning Π_{k,min} was defined as that haplotype partitioning with k SNPs that yielded the smallest relative residual deviance δ_{R}. The relationship between k and δ_{R}(Π_{k,min}), together with the number of haplotypes comprising Π_{k,min}, is depicted in FIG. 4. A qualitative difference was evident between k=6 and k=7 in that the number of haplotypes associated with Π_{k,min} increases from 13 to 22 whilst δ_{R}(Π_{k,min}) decreases only marginally [δ_{R}(Π_{6,min})=0.397 vs δ_{R}(Π_{7,min})=0.371]. It was therefore concluded that SNPs 1, 6, 7, 9, 11 and 14, which define Π_{6,min}, represented a good choice of key polymorphisms for further analysis. Of the remaining SNPs, six (nos. 3, 4, 8, 10, 12, and 16) could be classified as “marginally informative”. These markers, in combination with the six key SNPs, together define 39 of the 40 haplotypes observed, and account for virtually all of the explicable deviance (δ_{R}(Π_{12,min})=0.245). The other four SNPs (nos. 2, 5, 13 and 15) were “uninformative” with respect to the normalized in vitro expression level since they were either monomorphic in our sample (no. 2), or were in perfect (nos. 5 and 13) or near perfect (no. 15) linkage disequilibrium with other markers.

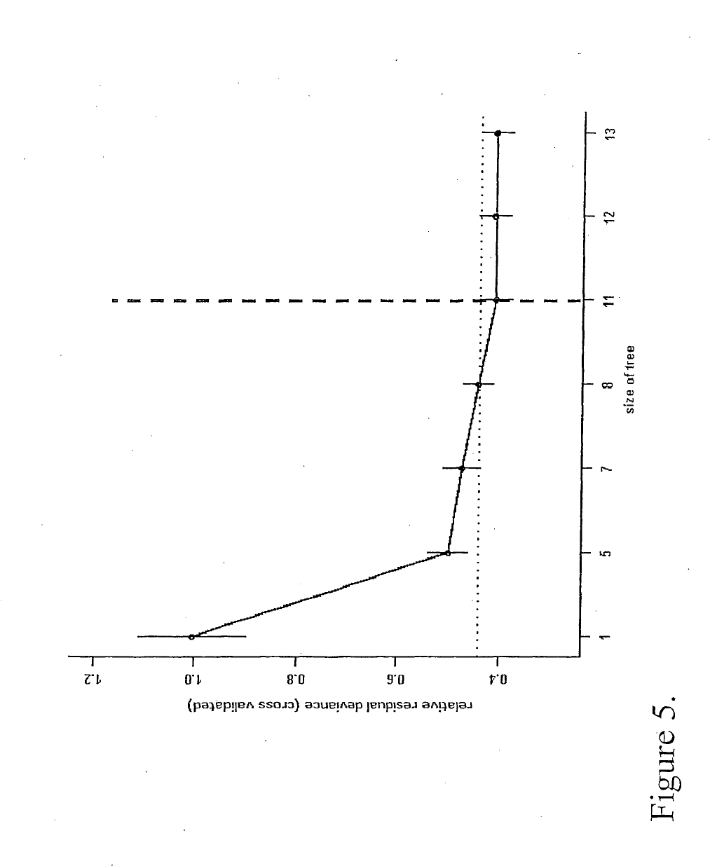

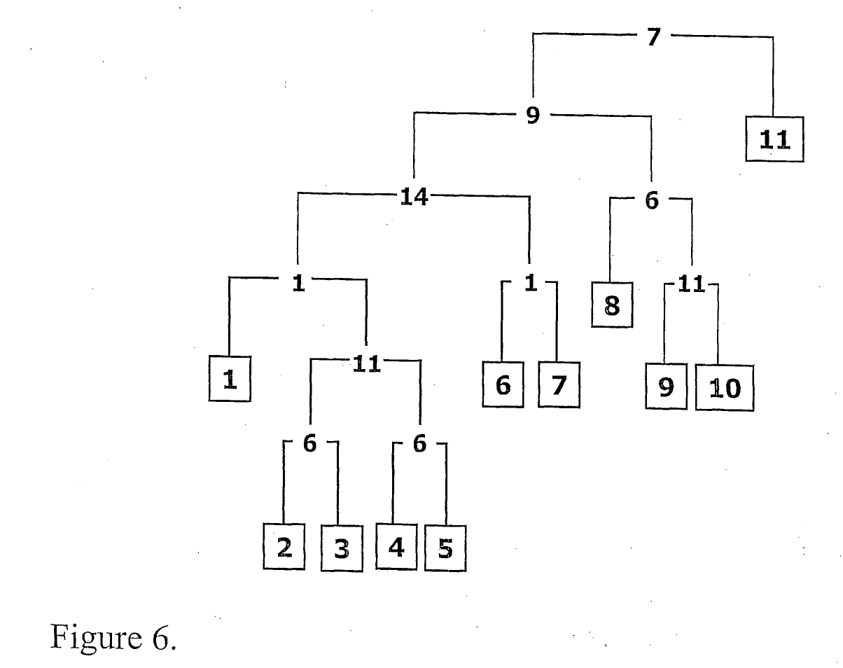

The correlation structure of the six key SNPs was next assessed using a series of successively growing (i.e. nested) regression trees. Following convention in regression tree analysis (Therneau and Atkinson 1997), the smallest intermediate tree with a cross-validated δ_{R} within one SE of that of the fully grown tree was chosen as a representative partitioning (FIG. 5). This ‘optimal’ tree was found to comprise 10 internal and 11 terminal nodes (FIG. 6, Table 5). The relative residual deviance of the tree equals δ_{R}=0.398, thereby accounting for (1−0.397)/(1−0.245)≈80% of the deviance explicable through haplotype partitioning.

The single most important split was by SNP 7 which on its own accounted for 15% of the explicable deviance. The four haplotypes carrying the C allele of this SNP define a homogeneous subgroup (leaf 11) with a mean normalized expression level 1.8 times higher than that of H1. Haplotypes carrying the T allele of SNP 7 were further sub-divided by SNP 9, with allele T of this polymorphism causing higher expression (μₙₒᵣ=1.26) than allele G (μₙₒᵣ=0.84; Wilcoxon z=7.09, p<0.001). The resulting nnTTnn haplotype was split by SNP 6 (G/T), with nGTTnn forming a terminal node (leaf 8) that includes the wild-type haplotype H1. Interestingly, the nTTTnn haplotypes, when sub-divided by SNP 11, manifested a dramatic difference in expression level. Whilst nTTTGn was found to be a low expresser (μₙₒᵣ=0.64), haplotype nTTTAn exhibited maximum average expression (μₙₒᵣ=3.89; Wilcoxon z=5.11, p<0.001).

Haplotype nnTGnn for SNPs 7 and 9 was sub-divided by SNPs 14 and 1, with three of the resulting haplotypes forming terminal nodes (leaves 1, 6 and 7). The fourth haplotype, GnTGnA, was an intermediate expresser (μₙₒᵣ=0.86) that was further split by SNPs 11 and 6. Interestingly, only one particular combination of SNP 14 and 1 alleles resulted in increased expression on the SNP 7 and 9 nnTGnn background (AnTGnG, leaf 7, μₙₒᵣ1.83). A similar non-additive effect upon expression was also noted for SNPs 6 and 11 when considered on haplotype GnTGnA: whereas SNP 11 allele A was associated with higher expression than G in combination with SNP 6 allele T (GTTGAA μₙₒᵣ=1.18 vs GTTGGA μₙₒᵣ=0.74; Wilcoxon z=7.09, p<0.001), the opposite held true in combination with SNP 6 allele G (GGTGAA μₙₒᵣ=0.74 vs GGTGGA μₙₒᵣ=1.04; Wilcoxon z=5.28, p<0.001).

Evolution of Haplotype Diversity

Of the 15 GH1 gene promoter SNPs found to be polymorphic in this study, alternative alleles at 14 positions were potentially explicable by gene conversion since they were identical to those in analogous locations in at least one of the four paralogous human genes (Table 3). Comparison with the orthologous growth hormone (GH) gene promoter sequences of 10 other mammals revealed that the most frequent alleles at nucleotide positions −75, −57, −31, −6, +3, +16 and +25 (corresponding to SNPs 8-15 inclusive) in the human GH1 gene were strictly conserved during mammalian evolution (Krawczak et al. 1999). Intriguingly, the rarest of the three alternative alleles at the −1 position (SNP 12) in the human GH1 gene was identical to that strictly conserved in the mammalian orthologues.

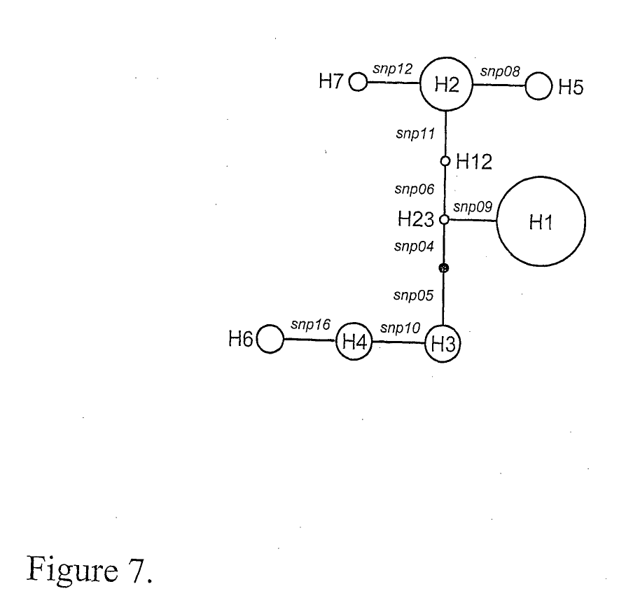

A ‘Reduced Median Network’ (FIG. 7) revealed that wild-type haplotype H1 is not directly connected to other frequent haplotypes by single mutational events. The second most common haplotype, H2, is connected to H1 via H23 and H12 whilst the third most common haplotype, H3, is connected to H1 either through a non-observed haplotype or a double mutation. Expansion of this network so as to incorporate further haplotypes was deemed unreliable owing to the small number of observations per haplotype. Furthermore, expansion of the network would have entailed the introduction of multiple single base-pair substitutions. Since these cannot be distinguished from serial rounds of gene conversion between pre-existing haplotypes, the resulting distances in the network would have been unlikely to reflect genuine evolutionary relationships. However, this may safely be assumed to be the case for the network depicted in FIG. 7 that connects the seven most frequent haplotypes, since each mutation occurs only once.

A general decline of linkage disequilibrium (LD) with physical distance was noted for most SNPs, with some notable exceptions (Table 6). Thus, SNP 9 was found to be in strong LD with the other SNPs, including SNP 16 which showed comparatively weak LD with all other proximal promoter SNPs. This finding suggests that the origin of SNP 9 was relatively late. However, SNP 10 was found to be in perfect LD with SNP 12 but not SNP 11 (ρ=0.381), whereas SNP 8 was in stronger LD with SNP 11 than with SNP 10 (ρ=0.925 vs 0.687). These anomalous findings suggest that the extant pattern of LD among the proximal promoter SNPs is unlikely to have arisen solely through recombinational decay with distance, but rather is likely to reflect the action of other mechanisms such as recurrent mutation, gene conversion or selection.

Prediction and Functional Testing of Super-Maximal and Sub-Minimal Haplotypes

Based upon the ‘optimal’ regression tree obtained for the haplotype-dependent proximal promoter expression data, an attempt was made to predict potential “super-maximal” and “sub-minimal” haplotypes in terms of their levels of expression. To this end, alleles of the six key SNPs were chosen taking the mean expression levels of the appropriate leaves of the tree into account (Table 5). Alleles of the remaining SNPs were determined so as to respectively maximize or minimize expression of individual SNPs. Thus, for the predicted super-maximal haplotype, alleles of SNPs 6, 7, 9 and 11 were as in leaf 10 whilst alleles of SNPs 1 and 14 were as in leaf 7. The sub-minimal haplotype was chosen to represent leaf 1 (for SNPs 1, 7, 9 and 14). The best choice of alleles for SNPs 6 and 11 was however somewhat ambiguous since leaves 2 (suggesting alleles T and G) and 4 (suggesting alleles G and A) predicted similarly low mean expression levels. Therefore, it was decided to generate both constructs for in vitro testing. Completion of the hypothetical haplotypes for the remaining SNPs yielded
super-maximal haplotype AGGGGTTAT-ATGGAG and
sub-minimal haplotypes AG-TTGTGGGACCACT, AG-TTTTGGGGCCACT.

These three artificial haplotypes were then constructed and expressed in rat pituitary cells yielding respectively expression levels of 145±4, 55±5 and 20±8% in comparison to wild-type (haplotype 1).

Differences Between SNP Alleles Revealed by Mobility Shift (EMSA) Assay

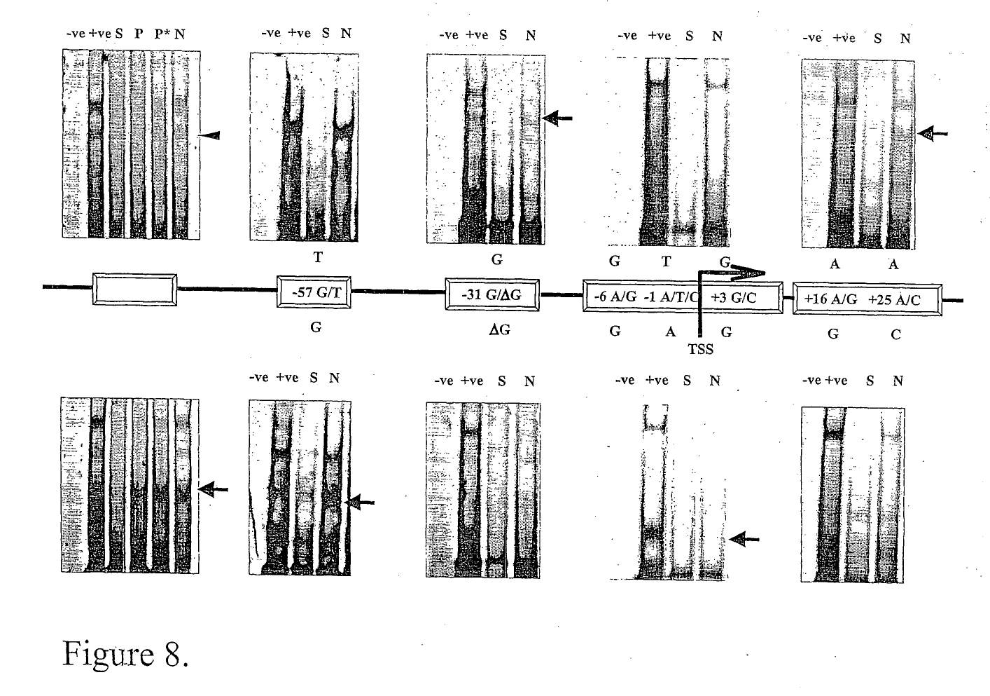

EMSAs were performed at all proximal promoter SNP sites for all allelic variants using rat pituitary cells as a source of nuclear protein. Protein interacting bands were noted at sites −168, −75, −57, −31, −6/−1/+3 and +16/+25 (Table 7). Inter-allelic differences in the number of protein interacting bands were noted for sites −75 (SNP 8), −57 (SNP 9), −31 (SNP 10), −6/−1/+3 (SNPs 11, 12, 13) and +16/+25 (SNPs 14, 15) [FIG. 8; Table 7]. In the case of the latter two sites, EMSA assays on specific SNP allele combinations suggested that differential protein binding was attributable to allelic variation at SNP sites 12 and 15 respectively (Table 7). When the analysis was repeated using a HeLa cell extract, only position −57 manifested evidence of a protein interaction and then only for the G allele, not the T allele (data not shown). The results of competition experiments utilizing oligonucleotides corresponding to two distinct Pit-1 binding sites were consistent with one of the two SNP 8 interacting proteins being Pit-1 (FIG. 8). However, the allele-specific protein interaction remained unaffected implying that the other protein involved was not Pit-1.

Association Between Promoter Haplotype Expression In Vitro and Stature In Vivo

An attempt was made to correlate the haplotype-specific in vitro expression of the GH1 proximal promoter with adult height in 124 male Caucasians. Each haplotype was ascribed its mean expression value from normalized in vitro expression data (Table 4) and the average Aₓ=(μ_{nor,h1}+μ_{nor,h2})/2 of the two haplotypes was calculated for each individual. Individuals homozygous for H1 were excluded from the analysis since their Aₓ values (1.0) would not have contributed any causal variation. This yielded a sample of 109 height-known individuals with suitable genotypes (Table 8). When height above and below the median (1.765 m) was compared to Aₓ values above and below the median (0.9), evidence for an association between height and GH1 proximal promoter haplotype-associated in vitro expression emerged (χ²=4.846, 1 d.f., P=0.028). This notwithstanding, regression analysis using a 2nd degree polynomial demonstrated that the two μₙₒᵣ values were on their own relatively poor predictors of height. Since the coefficient of determination was r²=0.025, it may be concluded that approximately 2.5% of the variance in body height is accounted for by reference to GH1 gene proximal promoter haplotype expression in vitro.

Locus Control Region (LCR) Polymorphisms and Proximal Promoter Strength

Three novel polymorphic changes were found within sites I and II (required for the pituitary-specific expression of the GH1 gene; Jin et al. 1999) of the GH1 LCR in a screen of 100 individuals randomly chosen from the study group. These were located at nucleotide positions 990 (G/A; 0.90/0.10), 1144 (A/C; 0.65/0.35) and 1194 (C/T; 0.65/0.35) [numbering after Jin et al. 1999]. The polymorphisms at 1144 and 1194 were in total linkage disequilibrium, and three different haplotypes were observed: haplotype A (990G, 1144A, 1194C, 0.55), haplotype B (990G, 1144C, 1194T; 0.35) and haplotype C (990A, 1144A, 1194C, 0.10).

In order to determine whether the three LCR haplotypes exert a differential effect on the expression of the downstream GH1 gene, a number of different LCR-GH1 proximal promoter constructs were made. The three alternative 1.6 kb LCR-containing fragments were cloned into pGL3, directly upstream of three distinct types of proximal promoter haplotype, viz. a “high expressing promoter” (H27), a “low expressing promoter” (H23) and a “normal expressing promoter” (H1), to yield nine different LCR-GH1 proximal promoter constructs in all. These constructs were then expressed in both rat GC cells and HeLa cells, and the resulting luciferase activities measured. In GC cells, the presence of the LCR enhances expression up to 2.8-fold as compared to the proximal promoter alone (Table 9). However, the extent of this inductive effect was dependent upon the linked promoter haplotype. Two-way analysis of variance (Table 10) revealed that both main effects and the promoter*LCR interaction were significant, with the major influence exerted by the proximal promoter. Also included in Table 9 are the results of a Tukey studentized range test at 95% significance level, performed individually for each promoter haplotype. In conjunction with promoter haplotype 1, the activity of LCR haplotype A is significantly different from that of N (construct containing proximal promoter but lacking LCR), but not from that of LCR haplotypes B and C; LCR haplotypes B and C differ significantly from each other and from N. With promoter 27, however, no significant difference was found between LCR haplotypes. No LCR-mediated induction of expression was noted with any of the proximal promoter haplotypes in HeLa cells (data not shown).

Since the physical distance between the LCR and the proximal promoter SNPs was too great to permit joint physical haplotyping, the linkage disequilbrium (LD) between them was assessed by maximum likelihood methods using genotype data from the 100 individuals included in the analysis of inter-SNP LD for the proximal promoter. Pair-wise LD between promoter SNPs and LCR haplotypes was found to be high for all SNPs except SNP 16 (Table 6). It may therefore be concluded that SNP 16 was subject to recurrent mutation prior to the genesis of SNP 9, the only SNP found to be in strong linkage disequilibrium with SNP 16. Substantial differences between LCR haplotypes exist in terms of their LD with SNPs 4, 8 and 16 (Table 6), suggesting a relatively young age for LCR haplotype B as opposed to haplotype A.

In our study we have determined that variation occurred at 15 of the 16 SNP locations within the proximal promoter of the GH1 gene manifesting itself in a total of 40 different promoter haplotypes. 12 haplotypes were found to be associated with a significantly reduced level of luceriferase reporter gene expression by comparison with haplotype 1, whereas 10 haplotypes were associated with the significantly increased level. Our data indicates that the conventional estimate of the variants in adult height attributable to polymorphic variation in the GH1 gene promoter (2.5%) is likely to be conservative and should be regarded as a minimum.

From the haplotype frequencies observed in our study group, it is predicted that some 8.2% of the normal population possess too low expressing GH1 proximal promoter haplotypes (either identical or non-identical) that are associated with in vitro GH production, that is equal or less than 50% of that of the wild-type.

Various cis acting regulatory sequences have been identified in the proximal promoter region of the growth hormone gene. Some of these factors may exert their effects synergistically whereas others appear to bind to promoter motifs in a mutually exclusive fashion. Inspection of the GH1 gene promoter region suggests that some of the 15 SNPs are located within transcription factor binding sites (FIG. 2). Thus, three SNPs cluster around the transcriptional initiation site (SNPs 11-13), one occurs at the 3′ end of the proximal VDRE adjacent to the TATA box (SNP 10), one within the distal VDRE (SNP 9), one within the proximal Pit-1 binding site (SNP 8) and one within an NF1 binding site (SNP 6). Expression analysis of a truncated promoter construct was consistent with a limited influence of SNPs 1-5 on GH1 gene expression.

Partitioning of the haplotypes identified 6 SNPs (numbers 1, 6, 7, 9, 11 and 14) as major determinants of GH1 gene expression level, with a further 6 SNPs being marginally informative (Nos. 3, 4, 8, 10, 12 and 16). The functional significance of all 16 SNPs was investigated by EMSA assays which indicated that 6 polymorphic sites in the GH1 proximal promoter interact with nucleic acid binding proteins; for 5 of these sites [SNP 8 (−75), 9 (−57), 10 (−31), 12 (−1) and 15 (+25)] alternative alleles exhibited differential protein binding.

Our study also focused on predicting potential super-maximal and sub-minimal haplotypes in terms of their expression levels. When tested, one of the sub-minimal haplotypes did manifest a lower level of expression than any naturally occurring haplotype, a result which indicates the efficacy of the process of haplotype partitioning described herein.

We hypothesised that the molecular bases for haplotype-dependent differences in GH1 gene promoter strength may thus lie in the net effect of the differential binding of multiple transcription factors to alternative versions of their cognate binding sites. The alternative versions of these sites differ by virtue of their containing different alleles of the various SNPs but combinatorially constitute the observed array of promoter haplotypes. The transcriptional activation of human genes is mediated by the interaction of transcription factors with different combinations and permutations of their cognate binding sites on the gene promoter. Some transcription factors are coordinated directly by cis-acting DNA sequence motifs, other indirectly by protein-protein interactions in what has been likened to a three-dimensional jigsaw puzzle: the DNA sequence motifs providing the puzzle template, the transcription factors constituting the puzzle pieces. This modular view of the promoter helps one to envisage how the effect of different SNP combinations in a given haplotype might be transfused so as to exert differential effects on transcription factor binding, transcriptosone assembly and hence gene expression. Thus, for example, the observed non-additive effects of GH1 promoter SNPs on gene expression may be understood in terms of the allele-specific differential binding of a given protein at 1-SNP site affecting, in turn, the binding of a second protein at another SNP site that is itself subject to allele-specific protein binding.

In our study, the LCR fragments serve to enhance the activity of the GH1 proximal promoter by up to 2.8-fold, although the degree of enhancement was found to be dependent upon the identity of the linked proximal promoter haplotype. Conversely, enhancement of the activity of a proximal promoter of given haplotype was also found to be dependent upon the identity of the LCR haplotype. Taken together, these findings imply that the genetic bases of inter-individual differences in GH1 gene expression is likely to be extremely complex.

Accordingly, our results demonstrate the significance of the haplotype in predicting the functionality of the nucleic acid molecule and so represents a useful stage in the analysis of genetic data.

TABLE 1
GH1 proximal promoter haplotypes defined by genetic variation at 16 locations

| | SNP position relative to GH1 gene transcriptional start site | | | | | | | | | | | | | | | | |
|---|---|---|---|---|---|---|---|---|---|---|---|---|---|---|---|---|---|
| No. | −476 | −364 | −339 | −308 | −301 | −278 | −168 | −75 | −57 | −31 | −6 | −1 | +3 | +16 | +25 | +59 | n |
| 1 | G | G | G | G | G | G | T | A | T | G | A | A | G | A | A | T | 103 |
| 2 | G | G | G | G | G | T | T | A | G | G | G | A | G | A | A | T | 50 |
| 3^{§} | G | G | G | T | T | G | T | A | G | G | A | A | G | A | A | T | 28 |
| 4^{§} | G | G | G | T | T | G | T | A | G | — | A | A | G | A | A | T | 16 |
| 5^{§} | G | G | G | G | G | T | T | G | G | G | G | A | G | A | A | T | 13 |
| 6 | G | G | G | T | T | G | T | A | G | — | A | A | G | A | A | G | 9 |
| 7^{§} | G | G | G | G | G | T | T | A | G | G | G | T | G | A | A | T | 8 |
| 8 | G | G | G | T | T | G | T | A | G | G | G | A | G | A | A | T | 6 |
| 9 | G | G | G | G | G | T | T | A | T | G | G | A | G | A | A | T | 6 |
| 10 | G | G | G | T | T | G | T | A | G | — | G | A | G | A | A | T | 6 |
| 11^{§} | G | G | G | G | G | T | T | G | G | G | G | A | G | G | C | T | 5 |
| 12 | G | G | G | G | G | T | T | A | G | G | A | A | G | A | A | T | 5 |
| 13^{§} | G | G | — | G | G | T | T | G | G | G | G | A | G | A | A | T | 5 |
| 14 | G | G | G | G | G | T | C | A | G | G | G | T | G | A | A | T | 5 |
| 15 | G | G | G | T | T | G | T | A | G | G | G | T | G | A | A | T | 4 |
| 16 | G | G | G | G | G | T | T | G | G | G | A | A | G | A | A | T | 4 |
| 17^{§} | G | G | — | G | G | T | T | A | G | G | G | A | G | A | A | T | 4 |
| 18 | G | G | G | G | G | T | T | A | G | — | G | A | G | A | A | T | 3 |
| 19^{§} | A | G | G | G | G | T | T | A | G | G | G | A | G | A | A | T | 3 |
| 20 | G | G | G | G | G | G | T | A | G | — | A | A | G | A | A | T | 3 |
| 21 | G | G | G | G | G | T | T | G | G | G | G | A | G | A | A | G | 3 |
| 22 | G | G | G | T | T | G | T | A | T | G | A | A | G | A | A | T | 3 |
| 23^{§} | G | G | G | G | G | G | T | A | G | G | A | A | G | A | A | T | 2 |
| 24^{§} | G | G | G | T | T | G | T | G | G | — | A | A | G | A | A | T | 2 |
| 25 | G | G | G | T | T | G | T | A | G | G | A | A | G | A | A | G | 1 |
| 26^{§} | G | G | G | G | G | T | T | G | G | G | G | T | G | A | A | T | 1 |
| 27 | G | G | G | G | G | T | T | A | T | G | A | A | G | A | A | T | 1 |
| 28 | G | G | G | G | G | T | T | A | G | — | A | A | G | A | A | T | 1 |
| 29^{§} | A | G | G | G | G | T | T | A | G | G | A | A | G | A | A | T | 1 |
| 30 | G | G | — | G | G | T | T | A | G | G | A | A | G | A | A | T | 1 |
| 31 | G | G | G | G | G | T | T | G | G | — | G | A | G | A | A | T | 1 |
| 32 | G | G | G | T | T | G | T | G | G | G | G | A | G | A | A | G | 1 |
| 33 | G | G | G | G | G | T | T | A | G | G | G | A | G | G | C | T | 1 |
| 34 | G | G | — | G | G | T | C | A | G | G | G | T | G | A | A | T | 1 |
| 35 | G | G | G | G | G | G | T | A | G | G | A | C | C | A | A | T | 1 |
| 36 | G | G | G | G | G | T | T | A | G | G | G | T | G | A | A | G | 1 |
| 37^{$} | A | G | G | G | G | T | T | A | G | G | G | A | G | G | A | T | 0 |
| 38^{$} | G | G | G | G | G | T | C | A | G | G | A | A | G | A | A | T | 0 |
| 39^{$} | G | G | G | T | T | G | T | A | G | G | G | A | G | A | C | T | 0 |
| 40^{$} | G | G | G | G | G | T | C | A | G | G | G | A | G | A | A | T | 0 |

n: frequency in 154 male British Caucasians;
^{§}haplotypes exhibiting a significantly reduced level (55% that of haplotype 1) of luciferase activity in GC cells;
^{$}only found in solitary cases of GH deficiency.
— denotes the absence of the base in question.

TABLE 2

Double-stranded oligonucleotide primer
sequences for EMSA analysis of SNP sites
exhibiting allele-specific protein
binding. SNP sites 11 - 15 were studied
in different allele combinations. TSS:
transcriptional initiation site.

| | Position | |
|---|---|---|
| SNP/allele | from TSS | Sequence 5′→3′ |
| 8 A | −89→−61 | CCATGCATAAATGTACACAGAAACAGGTG |
| | | CACCTGTTTCTGTGTACATTTATGCATGG |
| 8 G | | CCATGCATAAATGTGCACAGAAACAGGTG |
| | | CACCTGTTTCTGTGCACATTTATGCATGG |
| 9 G | −72→−42 | CAGAAACAGGTGGGGGCAACAGTGGGAGAGA |
| | | TCTCTCCCACTGTTGCCCCCACCTGTTTCTG |
| 9 T | | CAGAAACAGGTGGGGTCAACAGTGGGAGAGA |
| | | TCTCTCCCACTGTTGCCCCACCTGTTTCTG |
| 10 G | −45→−15 | GAGAAGGGGCCAGGGTATAAAAAGGGCCCAC |
| | | GTGGGCCCTTTTTATACCCTGGCCCCTTCTC |
| 10 ΔG | | GAGAAGGGGCCAGGTATAAAAAGGGCCCAC |
| | | GTGGGCCCTTTTTATACCTGGCCCCTTCTC |
| 11, 12, 13 | −18→+15 | CCACAAGAGACCAGCTCAAGGATCCCAAGGC |
| A A G | | CCGGGCCTTGGGATCCTTGAGCTGGTCTCTT |
| | | GTGG |
| 11, 12, 13 | | CCACAAGAGACCGGCTCAAGGATCCCAAGGC |
| G A G | | CCGGGCCTTGGGATCCTTGAGCCGGTCTCTT |
| | | GTGG |
| 11, 12, 13 | | CCACAAGAGACCGGCTCTAGGATCCCAAGGC |
| G T G | | CCGGGCCTTGGGATCCTAGAGCCGGTCTCTT |
| | | GTGG |
| 14, 15 | +4→+37 | ATCCCAAGGCCCAACTCCCCGAACCACTCAG |
| A A | | GGTACCCTGAGTGGTTCGGGGAGTTGGGCCT |
| | | TGGGAT |
| 14, 15 | | ATCCCAAGGCCCGACTCCCCGCACCACTCAG |
| G C | | GGTACCCTGAGTGGTGCGGGGAGTCGGGCCT |
| | | TGGGAT |
| 14, 15 | | ATCCCAAGGCCCGACTCCCCGAACCACTCAG |
| G A | | GGTACCCTGAGTGGTTCGGGGAGTCGGGCCT |
| | | TGGGAT |
| 14, 15 | | ATCCCAAGGCCCAACTCCCCGCACCACTCAG |
| A C | | GGTACCCTGAGTGGTGCGGGGAGTTGGGCCT |
| | | TGGGAT |

TABLE 3
Allele frequencies of 15 SNPs in the GH1 gene promoter
of 154 male Caucasians and corresponding nucleotides in
analogous locations of the paralogous genes of the GH cluster

| | GH1 | | | | | | |
|---|---|---|---|---|---|---|---|
| | Posi- | | | GH1 paralogues^{§} | | | |
| SNP | tion^{$} | Allele | Frequency | GH2 | CSH1 | CSH2 | CSHP1 |
| 1 | −476 | G | 304 (0.987) | A | G | G | A |
| | | A | 4 (0.013) | | | | |
| 3 | −339 | G | 297 (0.964) | G | G | G | G |
| | | — | 11 (0.036) | | | | |
| 4 | −308 | G | 232 (0.753) | T | C | C | T |
| | | T | 76 (0.247) | | | | |
| 5 | −301 | G | 232 (0.753) | T | T | T | T |
| | | T | 76 (0.247) | | | | |
| 6 | −278 | G | 185 (0.601) | T | A | A | T |
| | | T | 123 (0.399) | | | | |
| 7 | −168 | T | 302 (0.981) | T | C | C | T |
| | | C | 6 (0.019) | | | | |
| 8 | −75 | A | 273 (0.886) | G | A | A | G |
| | | G | 35 (0.114) | | | | |
| 9 | −57 | G | 195 (0.633) | A | T | T | G |
| | | T | 113 (0.367) | | | | |
| 10 | −31 | G | 267 (0.867) | — | G | G | G |
| | | — | 41 (0.133) | | | | |
| 11 | −6 | A | 181 (0.588) | A | G | G | A |
| | | G | 127 (0.412) | | | | |
| 12 | −1 | A | 287 (0.932) | A | T | T | C |
| | | T | 20 (0.065) | | | | |
| | | C | 1 (0.003) | | | | |
| 13 | +3 | G | 307 (0.997) | G | G | G | C |
| | | C | 1 (0.003) | | | | |
| 14 | +16 | A | 302 (0.981) | A | A | A | G |
| | | G | 6 (0.019) | | | | |
| 15 | +25 | A | 302 (0.981) | A | A | A | C |
| | | C | 6 (0.019) | | | | |
| 16 | +59 | T | 293 (0.951) | G | G | G | G |
| | | G | 15 (0.049) | | | | |

^{$}relative to the GH1 transcription start site;
^{§}bases at the analogous positions in the wild-type sequences of the four paralogous genes in the human GH cluster.

TABLE 4

In vitro GH1 gene promoter expression
analysis of 40 different SNP haplotypes

| | | | | |
|---|---|---|---|---|
| Haplotype | | | | |
| No. | n | μₙₒᵣ | σₙₒᵣ | Tukey |

| | | | | |
|---|---|---|---|---|
| 17 | 18 | 0.304 | 0.054 | a---------------- |
| 3 | 18 | 0.324 | 0.170 | a---------------- |
| 19 | 18 | 0.332 | 0.062 | a---------------- |
| 23 | 18 | 0.359 | 0.042 | ab--------------- |
| 24 | 18 | 0.395 | 0.107 | abc-------------- |
| 11 | 18 | 0.406 | 0.069 | abc-------------- |
| 26 | 18 | 0.410 | 0.181 | abc-------------- |
| 13 | 18 | 0.483 | 0.084 | abcd------------- |
| 29 | 18 | 0.502 | 0.149 | abcd------------- |
| 4 | 18 | 0.528 | 0.205 | abcde------------ |
| 5 | 18 | 0.536 | 0.205 | abcde------------ |
| 7 | 18 | 0.553 | 0.154 | abcdef----------- |
| 21 | 18 | 0.577 | 0.206 | * |
| 9 | 18 | 0.635 | 0.268 | abcdefg---------- |
| 15 | 18 | 0.725 | 0.271 | abcdefgh--------- |
| 25 | 18 | 0.790 | 0.229 | -bcdefghi-------- |
| 32 | 18 | 0.793 | 0.242 | -bcdefghi-------- |
| 33 | 18 | 0.807 | 0.225 | --cdefghi-------- |
| 35 | 18 | 0.809 | 0.230 | --cdefghi-------- |
| 18 | 12 | 0.819 | 0.217 | --cdefghi-------- |
| 10 | 18 | 0.855 | 0.135 | ---defghi-------- |
| 12 | 18 | 0.958 | 0.357 | ----efghij------- |
| 16 | 18 | 0.988 | 0.290 | -----fghijk------ |
| 1 | 90 | 1.000 | 0.174 | ------ghijk------ |
| 6 | 18 | 1.075 | 0.404 | -------hijkl----- |
| 2 | 18 | 1.078 | 0.150 | -------hijkl----- |
| 31 | 18 | 1.208 | 0.353 | --------ijklm---- |
| 28 | 18 | 1.317 | 0.312 | ---------jklmn--- |
| 8 | 18 | 1.333 | 0.453 | ---------jklmn--- |
| 22 | 18 | 1.403 | 0.380 | ----------klmno-- |
| 30 | 18 | 1.447 | 0.345 | -----------lmno-- |
| 36 | 18 | 1.451 | 0.368 | -----------lmno-- |
| 39 | 18 | 1.468 | 0.653 | -----------lmno-- |
| 20 | 18 | 1.600 | 0.342 | ------------mnop- |
| 38 | 18 | 1.697 | 0.752 | -------------nop- |
| 40 | 18 | 1.733 | 1.112 | * |
| 14 | 18 | 1.806 | 0.386 | --------------op- |
| 37 | 18 | 1.825 | 0.765 | --------------op- |
| 34 | 18 | 1.997 | 0.352 | ---------------p- |
| 27 | 18 | 3.890 | 0.901 | ----------------q |
| Negative | 90 | 0.000 | 0.005 | |
| control | | | | |

n: number of measurements; μₙₒᵣ: mean normalized expression level (i.e. fold change compared to H1); σₙₒᵣ: standard deviation of expression level; Tukey: result of Tukey's studentized range test, haplotypes with overlapping sets of letters are not statistically different in terms of their mean expression level; *: non-Gaussian distribution

TABLE 5
Haplotype partitioning of GH1 gene promoter expression data

| Haplotype^{§} | leaf^{&} | nₕₐₚ | n | μₙₒᵣ | σₙₒᵣ | δ(leaf) |
|---|---|---|---|---|---|---|
| nnCnnn | 11 | 4 | 72 | 1.809 | 0.725 | 36.27 |
| nGTTnn | 8 | 2 | 108 | 1.067 | 0.267 | 7.62 |
| nTTTGn | 9 | 1 | 18 | 0.635 | 0.268 | 1.22 |
| nTTTAn | 10 | 1 | 18 | 3.890 | 0.902 | 13.82 |
| AnTGnA | 1 | 2 | 36 | 0.418 | 0.142 | 0.71 |
| GnTGnG | 6 | 2 | 36 | 0.607 | 0.262 | 2.39 |
| AnTGnG | 7 | 1 | 18 | 1.825 | 0.765 | 9.95 |
| GTTGGA | 2 | 10 | 174 | 0.740 | 0.427 | 31.54 |
| GGTGAA | 4 | 8 | 144 | 0.735 | 0.474 | 32.16 |
| GGTGGA | 3 | 5 | 90 | 1.035 | 0.493 | 21.66 |
| GTTGAA | 5 | 4 | 72 | 1.178 | 0.384 | 10.47 |

nₕₐₚ: number of haplotypes included in leaf;
μₙₒᵣ: mean normalized expression level;
σₙₒᵣ: standard deviation of expression level;
δ(leaf): residual deviance within leaf;
^{§}alleles are given in the order of SNP 1, 6, 7, 9, 11 and 14 (n: any base);
^{&}numbering as in FIG. 4.

TABLE 6
Linkage disequilibrium, ρ, between GH1 proximal
promoter SNPs and LCR haplotypes in 100 male Caucasians

| | SNP | | | | | | | |
|---|---|---|---|---|---|---|---|---|
| SNP | 4 | 6 | 8 | 9 | 10 | 11 | 12^{&} | 16 |
| 4 | — | 1.000 | 0.802 | 0.893 | 0.731 | 0.554 | 0.638 | 0.567 |
| 6 | 1.000 | — | 0.927 | 0.868 | 0.632 | 0.891 | 0.867 | 0.111 |
| 8 | 0.802 | 0.927 | — | 1.000 | 0.687 | 0.925 | 0.242 | 0.251 |
| 9 | 0.893 | 0.868 | 1.000 | — | 1.000 | 0.905 | 1.000 | 1.000 |
| 10 | 0.731 | 0.632 | 0.687 | 1.000 | — | 0.381 | 1.000 | 0.415 |
| 11 | 0.554 | 0.891 | 0.925 | 0.905 | 0.381 | — | 1.000 | 0.044 |
| 12^{&} | 0.638 | 0.867 | 0.242 | 1.000 | 1.000 | 1.000 | — | 0.025 |
| 16 | 0.567 | 0.111 | 0.251 | 1.000 | 0.415 | 0.044 | 0.025 | — |
| LCR^{$} | 4 | 6 | 8 | 9 | 10 | 11 | 12 | 16 |
| A | 0.153 | 0.829 | 1.000 | 0.931 | 0.601 | 0.782 | 0.800 | 0.064 |
| B | 1.000 | 0.952 | 0.922 | 0.958 | 0.531 | 0.873 | 0.831 | 0.643 |
| C | 0.840 | 0.997 | 0.491 | 0.840 | 0.875 | 0.482 | 1.000 | 0.289 |

^{&}a single chromosome out of 200 was found to carry SNP12 allele C; this chromosome was excluded from all LD analyses involving SNP12;
^{$}for each LCR haplotype, ρ was calculated against the combination of the other two LCR haplotypes, thereby turning the LCR into a biallelic system.

TABLE 7
Results of EMSA assays that demonstrated allele-specific
differential protein binding at the various SNP sites in the
GH1 gene promoter using rat pituitary cell nuclear extracts.

| | Position of | | No. of protein | | | Transcription factor |
|---|---|---|---|---|---|---|
| | double-stranded | Sequence | interacting bands | | | binding site/ |
| SNP | oligonucleotide | variation | Strong | Medium | Weak | functional region |
| 8 | −89 → −61 | −75 A | — | 1 | — | Pit-1 |
| | | −75 G | 1 | 1 | — | Pit-1 |
| 9 | −72 → −42 | −57 T | 1 | — | — | Vitamin D receptor |
| | | −57 G | 2 | — | — | Vitamin D receptor |
| 10 | −45 → −15 | −31 G | 1 | — | — | TATA box |
| | | −31 ΔG | — | — | 1 | TATA box |
| 11, 12, 13 | −18 → +15 | −6/−1/+3 | — | — | — | TSS |
| | | AAG | | | | |
| | | −6/−1/+3 | — | — | — | TSS |
| | | GAG | | | | |
| | | −6/−1/+3 | 1 | — | — | TSS |
| | | GTG | | | | |
| 14, 15 | +4 → +37 | +16/+25 | 2 | 1 | — | 5′UTR |
| | | AA | | | | |
| | | +16/+25 | 2 | — | — | 5′UTR |
| | | AC | | | | |
| | | +16/+25 | 1 | — | — | 5′UTR |
| | | GC | | | | |
| | | +16/+25 | 2 | 1 | — | 5′UTR |
| | | GA | | | | |

TSS: Transcriptional start site
5′UTR: 5′ untranslated region

TABLE 8
Association between adult height and GH1 proximal promoter
haplotype-associated in vitro expression data in 124 male Caucasians

| | Aₓ < 0.9 | Aₓ > 0.9 |
|---|---|---|
| height < 1.765 | 34 | 22 |
| height > 1.765 | 21 | 32 |

Aₓ: average normalized in vitro expression level of the two haplotypes of an individual i.e. Aₓ = (μ_{nor,h1} + μ_{nor,h2})/2.

TABLE 9
Average GC cell-derived, normalized luciferase activities ± standard
deviation of different LCR-GH1 proximal promoter constructs

| Promoter | LCR haplotype | | | |
|---|---|---|---|---|
| haplotype | N | A | B | C |
| H1 | 1.00 ± 0.26^{x} | 2.47 ± 0.41^{yz} | 2.30 ± 0.46^{y} | 2.77 ± 0.55^{z} |
| H23 | 1.00 ± 0.14^{x} | 1.72 ± 0.55^{yz} | 2.14 ± 0.52^{z} | 1.35 ± 0.48^{xy} |
| H27 | 1.00 ± 0.26^{x} | 1.11 ± 0.36^{x} | 1.00 ± 0.41^{x} | 1.25 ± 0.27^{x} |

^{x,y,z}Tukey's studentized range test within a promoter haplotype;
LCR haplotypes (A, B and C) with overlapping sets of letters are not statistically different in terms of their mean expression level.
N: Construct containing proximal promoter but lacking LCR.
LCR haplotypes were normalised with respect to N in each case.

TABLE 10
Two-way ANOVA of normalized luciferase activities of LCR-GH1
proximal promoter constructs

| Source | DF | Mean Square | F Value | Pr > F |
|---|---|---|---|---|
| Promoter haplotype | 2 | 51.46 | 390.97 | <0.0001 |
| LCR haplotype | 3 | 5.67 | 43.08 | <0.0001 |
| Interaction | 6 | 3.09 | 23.48 | <0.0001 |

1-8. (canceled)

9. A method for identifying at least one mutation and/or polymorphism that is a major determinant of a phenotype, comprising calculating a residual deviance (δ) of a predetermined group of mutations and/or polymorphisms identified within a gene.

10. The method according to claim 9, wherein the residual deviance (δ) is calculated for each possible subset of said predetermined group of mutations and/or polymorphisms.

11. The method according to claim 10, wherein said each possible subset of mutations and/or polymorphisms is defined by partitioning a predetermined group of haplotypes {1 . . . m} corresponding to said mutations and/or polymorphisms.

12. The method according to claim 11, wherein the residual deviance (δ) is calculated as δ=δ(Π)=Σᵢ₌₁^{m}(χᵢ=\overline{χ}_{Π(i)})^{2.}

13. A method for predicting a super-maximal and/or a sub-minimal haplotype that are major determinants of a corresponding super-maximal phenotype and a sub-minimal phenotype, comprising calculating a residual deviance (δ) of said super-maximal and/or sub-minimal haplotype in accordance with the method of claim 11.

14. A method for identifying at least one single nucleotide polymorphism (SNP) that is of phenotypic significance, comprising calculating a residual deviance (δ) of said at least one single nucleotide polymorphism (SNP) in accordance with the method of claim 11.

15. A detection method for detecting a haplotype effective to act as an indicator of at least one phenotype in an individual, which detection method comprises the steps of:
(a) obtaining a test sample of genetic material from an individual to be tested, said material comprising at least a selected gene or a fragment thereof;
(b) analysing a nucleotide sequence of said gene or fragment thereof to determine whether single nucleotide polymorphisms (SNPs) exist at any one or more of the SNP sites within the gene; and
(c) where said SNPs exist, identifying them in order to determine a haplotype of said individual; and
(d) calculating a residual deviance (δ) of said haplotype in accordance with the method of claim 11.

16. A method for identifying a haplotype which is phenotypically significant in the diagnosis or treatment of a disease characterised by said phenotype, comprising calculating a residual deviance (δ) of said haplotype in accordance with the method of claim 11.


SEQUENCE LISTING (40 sequences)

SEQ ID NO: 1 - DNA, 16 bp, human
ggggggtatgaagaat

SEQ ID NO: 2 - DNA, 16 bp, human
gggggttagggagaat

SEQ ID NO: 3 - DNA, 16 bp, human
gggttgtaggaagaat

SEQ ID NO: 4 - DNA, 16 bp, human
  misc_feature (10)..(10): note = n may be any nucleotide
gggttgtagnaagaat

SEQ ID NO: 5 - DNA, 16 bp, human
gggggttggggagaat

SEQ ID NO: 6 - DNA, 16 bp, human
  misc_feature (10)..(10): note = n may be any nucleotide
gggttgtagnaagaag

SEQ ID NO: 7 - DNA, 16 bp, human
gggggttagggtgaat

SEQ ID NO: 8 - DNA, 16 bp, human
gggttgtagggagaat

SEQ ID NO: 9 - DNA, 16 bp, human
gggggttatggagaat

SEQ ID NO: 10 - DNA, 16 bp, human
  misc_feature (10)..(10): note = n may be any nucleotide
gggttgtagngagaat

SEQ ID NO: 11 - DNA, 16 bp, human
gggggttggggaggct

SEQ ID NO: 12 - DNA, 16 bp, human
gggggttaggaagaat

SEQ ID NO: 13 - DNA, 16 bp, human
  misc_feature (3)..(3): note = n may be any nucleotide
ggnggttggggagaat

SEQ ID NO: 14 - DNA, 16 bp, human
gggggtcagggtgaat

SEQ ID NO: 15 - DNA, 16 bp, human
gggttgtagggtgaat

SEQ ID NO: 16 - DNA, 16 bp, human
gggggttgggaagaat

SEQ ID NO: 17 - DNA, 16 bp, human
  misc_feature (3)..(3): note = n may be any nucleotide
ggnggttagggagaat

SEQ ID NO: 18 - DNA, 16 bp, human
  misc_feature (10)..(10): note = n may be any nucleotide
gggggttagngagaat

SEQ ID NO: 19 - DNA, 16 bp, human
aggggttagggagaat

SEQ ID NO: 20 - DNA, 16 bp, human
  misc_feature (10)..(10): note = n may be any nucleotide
ggggggtagnaagaat

SEQ ID NO: 21 - DNA, 16 bp, human
gggggttggggagaag

SEQ ID NO: 22 - DNA, 16 bp, human
gggttgtatgaagaat

SEQ ID NO: 23 - DNA, 16 bp, human
ggggggtaggaagaat

SEQ ID NO: 24 - DNA, 16 bp, human
  misc_feature (10)..(10): note = n may be any nucleotide
gggttgtggnaagaat

SEQ ID NO: 25 - DNA, 16 bp, human
gggttgtaggaagaag

SEQ ID NO: 26 - DNA, 16 bp, human
gggggttggggtgaat

SEQ ID NO: 27 - DNA, 16 bp, human
gggggttatgaagaat

SEQ ID NO: 28 - DNA, 16 bp, human
  misc_feature (10)..(10): note = n may be any nucleotide
gggggttagnaagaat

SEQ ID NO: 29 - DNA, 16 bp, human
aggggttaggaagaat

SEQ ID NO: 30 - DNA, 16 bp, human
  misc_feature (3)..(3): note = n may be any nucleotide
ggnggttaggaagaat

SEQ ID NO: 31 - DNA, 16 bp, human
  misc_feature (10)..(10): note = n may be any nucleotide
gggggttggngagaat

SEQ ID NO: 32 - DNA, 16 bp, human
gggttgtggggagaag

SEQ ID NO: 33 - DNA, 16 bp, human
gggggttagggaggct

SEQ ID NO: 34 - DNA, 16 bp, human
  misc_feature (3)..(3): note = n may be any nucleotide
ggnggtcagggtgaat

SEQ ID NO: 35 - DNA, 16 bp, human
ggggggtaggaccaat

SEQ ID NO: 36 - DNA, 16 bp, human
gggggttagggtgaag

SEQ ID NO: 37 - DNA, 16 bp, human
aggggttagggaggat

SEQ ID NO: 38 - DNA, 16 bp, human
gggggtcaggaagaat

SEQ ID NO: 39 - DNA, 16 bp, human
gggttgtagggagact

SEQ ID NO: 40 - DNA, 16 bp, human
gggggtcagggagaat